(19) United States
(12) Patent Application Publication
(10) Pub. No.: US 2009/0274738 A1
(43) Pub. Date: Nov. 5, 2009

(54) IMPLANTABLE DEVICES FOR PROMOTING REENDOTHELIALIZATION AND METHODS OF USE THEREOF

(76) Inventor: Jean-Francois TANGUAY, Montreal (CA)

Correspondence Address: ROTHWELL, FIGG, ERNST & MANBECK, P.C., 1425 K STREET, N.W., SUITE 800, WASHINGTON, DC, 20005, US

(21) Appl. No.: 12/113,570

(22) Filed: May 1, 2008

(51) Int. Cl. A61F 2/00

(52) U.S. Cl. 424/423

(57) ABSTRACT

An implantable device for the controlled delivery of an estrogen receptor agonist to an injured site in the lumen of a mammalian blood vessel, wherein the estrogen receptor agonist is present in an amount of at least about 16.7 μg/mm implantable device length. Methods of use thereof.

### FIELD OF THE INVENTION

The present invention relates to implantable devices for promoting reendothelialization and methods of use thereof. More specifically, the present invention is concerned with implantable devices for the controlled delivery of an estrogen receptor agonist at an injured site of a injured vessel.

### BACKGROUND OF THE INVENTION

17-β-Estradiol is known to inhibit proliferation of smooth muscle cells (neointima formation) and promote reendothelialization in vitro and in vivo. Co-pending application Ser. No. 10/088,405 has shown that estradiol inhibits restenosis and promotes reendothelialization in a pig model for restenosis.

Results of administering a bolus of either 600 μg (co-pending application Ser. No. 10/088,405 and Chandrasekar B et al. J Am Coll Cardio 2001; 38(5):1570-6) or 100 ug/kg and 200 ug/kg (total of 2 to 4 mg) (Chandrasekar B et al. Thromb Haemost 2005; 94:1042-7) by catheter at the site of the injury are known. With such a type of administration, it is generally believed that less than about 1% of the administered dosage remains on site for 24 hours so that it is assumed that for such an administered amount, about 6 to 40 μg remained on site. This dosage was based on the dosage administered sublingually in postmenopausal women. The patent application suggested that it may be unnecessary to use doses as high as 4 mg for local administration and further suggested that bolus doses of 600 μg had been tried and was found to be as effective as the dose of 2 or 4 mg to reduce neointima formation.

These experiments did not suggest optimal dosage for controlled delivery administration of estrogen receptor agonist by implantable device.

Administration of 17 beta-estradiol on a stent was also tested at different dosages.

U.S. Pat. No. 6,471,979 to Estrogen Vascular Technology, LLC reported results of preclinical trials testing the safety and efficacy of 17 beta-estradiol BiodivYsio™ phosphorylcholine (PC) coated eluting stainless steel stents. Low doses of 67 μg on 18 mm stents (about 3.72 μg per mm of stent) and high doses of 229 μg to 276 μg on 18 mm stents (about 12.72-15.33 μg per mm of stent (or about 2.4-3.2 μg/mm²)) had been administered to pigs on phosphorylcholine coated stainless steel stents. Reduced intimal area and partial reendothelialization had then been obtained. With those stents, the percentage of released estradiol uptaken by the coronary tissue was not determined.

Clinical trials were then undertaken which were reported in US 2004/0127475 also to Estrogen Vascular Technology, LLC. BiodivYsio™ phosphorylcholine coated stainless steel stents with a dose of 2.52 μg per mm² of stent (i.e. about 250 μg per stent with 18 mm×3 mm stents namely about 13.88 μg per mm of length of stent. Two patients suffered in-stent stenosis (>50% diameter stenosis) and the remaining patients had a mean neointimal volume obstruction of 23.5±12.5% (i.e. 32.3±16.4 mm³ with a stent volume of 143.7 5±43.7 mm³) (Abizaid A et al. JACC 2004; 43 (6):1118-21). The 6 months follow up showed a low amount of intimal hyperplasia and late-loss, and only one out of 30 patients required target vessel revascularization. Nevertheless, neointimal proliferation was not completely abolished by these estradiol-eluting stents. These unsatisfying results were attributed to the suboptimal estradiol elution of their delivery system which provided an elution that stopped within 24 hours after administration.

Estrogen Vascular Technology, LLC. conducted another clinical trial (Ethos I) using a biostable polymer (PEVA/PBMA Bravo™) coated R™ stent (open cell, flexible) loaded with 16 μg/mm stents (240 μg on a 15 mm stent coated with 564 μg of biostable polymer) coated with fast-release and moderate-release formulations. 95 patients were enrolled (32 with the bare metal stent (BM), 31 with the fast release and 32 with the moderate release). The release profile of the moderate release formulations measured in porcine coronary artery was expected to be of 28 days. Although this trial again confirmed that there were no safety concerns with estradiol administration (i.e. death, or stent thrombosis), the results were again disappointing. There was no evidence of benefit over bare metal stent associated with the estradiol-eluting stents in clinical, QCA, and IVUS assessments at 6 months follow up. Estrogen Vascular Technology, LLC. reported preclinical trials results which revealed that estradiol enhanced endothelial progenitor cell growth between 10 nM and 100 nM concentration but has a strong negative effect higher than 10 μM and above and that estradiol enhanced foetal bovine aortic endothelial cell growth between 0.1 nM and 10 nM concentration but that this effect was lost at 100 nM and above. In view of this, Estrogen Vascular Technology, LLC. planned to conduct additional trials with decreased estradiol doses. Pre-clinical testing suggested that estradiol is more likely to produce beneficial effects at lower doses. Estrogen Vascular Technology, LLC. reported that it expected that its future experiments using a dosage of 75 μg on a 15 mm stent (i.e. 5 μg/mm) (Ethos II) coated with 282 μg biostable polymer would yield better results and using a dosage of 20 μg on a 15 mm stent (i.e. 1.33 μg/mm) (Ethos III) coated with 80 μg of a degradable abluminal polymer would yield even better results. After 6 months, Ethos II showed no effect when compared to BMS. To the Applicant's knowledge no results were reported for the Ethos III trials.

Late stent thrombosis generally refers to thrombosis that occurs at least one month following stent implantation, while very late stent thrombosis generally refers to events that occur more than 12 months following stent placement (Hodgson J. et al. Cardiovascular Interventions 2007; 69:327-33). It has been reported that delayed or incomplete reendothelialization is likely a cause of late susceptibility to stent thrombosis. In particular, there is a reported increase of late stent thrombosis with both sirolimus-eluting and paclitaxel-eluting stents between 1 and 4 years of follow-up. It has also been reported that a stabilized neointima is not a negative parameter so long as the artery lumen is not reduced below a certain threshold (Finn A et al. Circ 2007; 115 :2435-41, Kotani et al. J. Am. Coll Cardiol 2006; 47 :2108-11).

Thus, there is a need for an agent that would promote reendothelialization and for an improved dosage for in situ administration of an estradiol receptor agonist.

The present description refers to a number of documents, the content of which is herein incorporated by reference in their entirety.

### SUMMARY OF THE INVENTION

The present invention is concerned with the surprising finding that administration of a dosage of estrogen receptor agonist of at least 16.7 μg/mm of implantable device at the injured site of a vessel accelerates and optimizes vessel repair.

With the device of the present invention, after the first 24 hours, a mean detected concentration of 13.6 μg estradiol/g of coronary tissue with a maximum of 50 μg estradiol/g of coronary tissue could be detected in the tissue post implantation using the method GC/MS. For a stent covered with 500 μg estradiol, this represents 2.7% of the original dose. In contrast, 0.51% of 10 mg (i.e. 51 μg in the tissue overall) was up-taken by the tissue when administered with a catheter over a few seconds and was reduced to a residual amount (i.e. slightly over background noise) within an hour. The mean detected concentration during the first 48 hours was up to 12 μg estradiol/g of coronary tissue and during the first week (168 hours) of up to 10.6 μg estradiol/g of coronary tissue. Reaching as fast as possible an effective dose for reendothelialization in the tissue is desirable since early reendothelialization reduces the risk of restenosis.

More specifically, in accordance with an aspect of the present invention, there is provided an implantable device for the controlled delivery of an estrogen receptor agonist to an injured site in the lumen of a mammalian blood vessel, wherein the estrogen receptor agonist is present in an amount of at least about 16.7 μg/mm implantable device length.

In a specific embodiment of the implantable device of the present invention, the blood vessel is procedurally traumatized. In another specific embodiment of the implantable device of the present invention, the estrogen receptor agonist is present in an amount of at least about 21.7 μg/mm of device length.

In another specific embodiment of the implantable device of the present invention, the estrogen receptor agonist is present in an amount of at least about 27.8 μg/mm of device length. In another specific embodiment of the implantable device of the present invention, the estrogen receptor agonist is present in an amount of at least about 30.8 μg/mm of device length. In another specific embodiment of the implantable device of the present invention, the estrogen receptor agonist is present in an amount of at least about 49.5 μg/mm of device length. In another specific embodiment of the implantable device of the present invention, the controlled delivery enables a mean concentration of estrogen receptor agonist in coronary tissue of at least about 9.1 μg/g of coronary tissue over at least about 2 weeks. In another specific embodiment of the implantable device of the present invention, the controlled delivery enables a mean concentration of estrogen receptor agonist in coronary tissue of at least about 10.6 μg/g of coronary tissue over at least about 1 week. In another specific embodiment of the implantable device of the present invention, the controlled delivery enables a mean concentration of estrogen receptor agonist in coronary tissue of at least about 12.0 μg/g of coronary tissue over at least about 48 hours. In another specific embodiment of the implantable device of the present invention, the controlled delivery enables a mean concentration of estrogen receptor agonist in coronary tissue of at least about 13.6 μg/g of coronary tissue over at least about 24 hours. In another specific embodiment of the implantable device of the present invention, the controlled delivery enables a mean concentration of estrogen receptor agonist in coronary tissue of at least about 50 μg/g of coronary tissue over at least about 24 hours.

In another specific embodiment, the implantable device of the present invention is a stent. In another specific embodiment, the implantable device of the present invention is a shunt. In another specific embodiment, the implantable device of the present invention is a mesh. In another specific embodiment, the implantable device of the present invention is an artificial graft.

In another specific embodiment of the implantable device of the present invention, the estrogen receptor agonist is 17-β estradiol. In another specific embodiment of the implantable device of the present invention, the estrogen receptor agonist is releasably embedded in, coated on, or embedded in and coated on, the device.

In another specific embodiment of the implantable device of the present invention, the device further comprises a controlled release polymer coating. In another specific embodiment, the polymer coating is biodegradable. In another specific embodiment of the implantable device of the present invention, the 17-β estradiol amount is sufficient to allow complete reendothelialization of the injured site.

In another specific embodiment, the implantable device of the present invention further comprises an agent selected from the group consisting an antioxidant, an anti-proliferative/cytostatic agent, an anti-inflammatory/immunomodulator agent; an anti-migration agent, a pro-healing agent, and combinations thereof.

In another specific embodiment, the cytostatic agent is paclitaxel or an analog thereof. In another specific embodiment, the cytostatic agent is rapamycin or an analog thereof. In another specific embodiment, the cytostatic agent is sirolimus or an analog thereof. In another specific embodiment, the anti-migration agent inhibits migration of vascular smooth muscle cells. In another specific embodiment, the inhibitor of migration of vascular smooth muscle cells is cytochalasin.

In another specific embodiment of the implantable device of the present invention, the mammalian blood vessel is a human blood vessel.

In accordance with another aspect of the present invention, there is provided a method for promoting reendothelialization of an injured blood vessel of a subject, comprising implanting the implantable device of the present invention at an injured site of the blood vessel of the subject, whereby reendothelialization is promoted.

In an specific embodiment of the method of the present invention, the subject is human.

In an specific embodiment of the method of the present invention, the device is implanted before, during or after vascular injury, or any combination thereof.

In an specific embodiment of the method of the present invention, the injured mammalian blood vessel is a procedurally traumatized blood vessel.

In accordance with another aspect of the present invention, there is provided a use of the implantable device of the present invention for promoting reendothelialization of an injured blood vessel of a subject.

### DEFINITIONS

As used herein the terms “estradiol receptor agonist” refer to estradiol such as 17 beta estradiol, 17 alpha estradiol and their hydroxylated metabolites with or without subsequent glucuronidation, sulfation, esterification or O-methylation; an estradiol precursor; an active estradiol metabolite such as estrone and estriol; an active analog such as mycoestrogens and phytoestrogens including coumestans, prenylated flavonoid, isoflavones (e.g. genistein, daidzein, biochanin A, formononetin and coumestrol), and ligands; a modulator capable of positively influencing the activity of the estradiol receptor(s) or of enhancing the binding and/or the activity of estradiol towards its receptor such as a selective estrogen receptor modulator (SERM) including tamoxifen and a derivative thereof including clomifene, raloxifene, toremifene, bazedoxifene, lasofoxifene, ormeloxifenem, tibolone and idoxifene; a selective estrogen receptor down-regulator (SERD) including sulvestrant, ethamoxytriphetol and nafoxidine; and a high dose estradiol such as diethylstilbestrol and ethinyloestradiol; testosterone. Dehydroepiandrosterone (DHEA) is produced from cholesterol through two cytochrome P450 enzymes. Cholesterol is converted to pregnenolone by the enzyme P450 scc (side chain cleavage) and then another enzyme CYP17A1 converts pregnenolone to 17α-Hydroxypregnenolone and then to DHEA. In humans, DHEA is the dominant steroid hormone and the precursor of all sex steroids. After side chain cleavage, and either utilizing the delta-5 pathway or the delta-4 pathway, androstenedione is another key intermediary. Androstenedione is either converted to testosterone, which in turn undergoes aromatization to estradiol, or, alternatively, androstenedione is aromatized to estrone which is converted to estradiol. As used herein, the terms estradiol precursor include androstenedione and estrone.

As used herein the terms “injured mammalian blood vessel” refer to a procedurally traumatized blood vessel, and to a blood vessel affected by arterial injuries that are not the result of a clinical procedure. Without being so limited the terms include blood vessels affected by stenosis, restenosis, high risk plaque and vulnerable plaque.

As used herein the terms “procedurally traumatized mammalian blood vessel” refer to a vessel injured by a surgical/mechanical/cryotherapy/laser intervention into mammalian vasculature. Without being so limited procedural traumas include organ transplantation, such as heart, kidney, liver and the like, e.g., involving vessel anastomosis; vascular surgery, e.g., coronary bypass surgery, biopsy, heart valve replacement, atherectomy, thrombectomy, and the like; transcatheter vascular therapies (TVT) including angioplasty, e.g., laser angioplasty and PTCA procedures, employing balloon catheters, and indwelling catheters; vascular grafting using natural or synthetic materials, such as in saphenous vein coronary bypass grafts, dacron and venous grafts used for peripheral arterial reconstruction, etc.; placement of a mechanical shunt, e.g., a PTFE hemodialysis shunt used for arteriovenous communications; and placement of an intravascular stent, which may be metallic, plastic or a biodegradable polymer.

As used herein the terms “delivery system” includes without being so limited implantable devices, perivascular gels, microspheres and micelles.

The implantable device of may further comprise at least one further agent selected from the group consisting an antioxidant such as but not limited to nitric oxide; an anti-proliferative or cytostatic agent such as but not limited to paclitaxel zotarolimus, biolimus, statin, mitomycin, actinomycin, C-myc antisens, restenase, PCNA ribozyme, 2-chlorodeoxyadenosine, vincristine, methothrexate, angiopeptin or a PDGF inhibitor; an anti-inflammatory/immunomodulator agent such as but not limited to cyclosporine, Interferon γ-1b, dexamethasone, leflunomide, sirolimus, tacrolimus, everolimus, mycophenolic acid, mizorbine or tranilast; an anti-migration agent such as but not limited to batimastat, MMP inhibitor, probucol, prolyl hydroxylase inhibitors, cytochalasin, halofuginone or C-proteinase inhibitors; and a pro-healing agent such as VEGF, an EPC antibody and biorest. Analogs or derivatives of the listed agents can also be used. For instance the paclitaxel derivative docetaxel can also be used.

As used herein the terms “implantable device” refers to, without being so limited, stent, shunt, mesh (membrane polymer, intracoronary, endocardiac, epicardiac) and graft made of natural or synthetic materials.

As used herein the terms “injured site” when used to refer to an injured site in a vessel refer to the site of injury or upstream of the injury.

As used herein the terms “biodegradable polymer” refer to a polymer that is biocompatible with target tissue and the local physiological environment into which the dosage form to be administered and capable of being decomposed into biocompatible products by natural biological processes. Such polymers degrade over a period of time preferably between from about 48 hours to about 180 days, preferably from about 1-3 to about 150 days, or from about 3 to about 180 days, or from about 10 to about 30 days. Without being so limited, biodegradable polymers encompassed by the present invention include polylactic acid (PLLA), polyglycolic acid (PGA), polyesthers, polyanhydride, polyiminocarbonate, inorganic calcium phosphate, polycaprolactone (PCL), aliphatic polycarbonates, polyphosphazenes, phosphorylcholine-based, hydroxybutarate valerate, and polyethyleneoxide/polybutylene terepthalate.

As used herein the terms “effective amount of biodegradable polymer” refers to an amount of polymer that enables the loading of as much estrogen receptor agonist as possible in accordance with the present invention. The precise amount of polymer thus depends on its nature and on the nature of the estrogen receptor agonist. Polymers such as PEA from Medivas enables the loading of therapeutic agent in an amount about equal to its own weight (e.g. for 500 μg of polymer, up to 500 μg of estrogen receptor agonist can be loaded). A top coat of polymer can also be applied in addition to this amount to decrease release speed. The present invention also encompasses the chemical coupling of the estradiol receptor agonist to the polymer to slow down its release from this polymer.

As used herein the terms “controlled release” is meant to refer to gradual release as opposed to immediate release. This type of release can be achieved with or without a polymer coating. Without being so limited, a controlled release without a polymer can be achieved with a Translumina™ stent.

As used herein the terms “controlled release polymer coating” refers to a polymer coating that dispenses the therapeutic agent that it contains in the body gradually. It includes delayed release, fast and slow release.

As used herein the terms “biological system” refers to a cell or cells, a tissue or a subject.

As used herein the term “subject” is meant to refer to any mammal including human, mice, rat, dog, cat, pig, cow, monkey, horse, etc. In a particular embodiment, it refers to a human.

As used herein the term “coronary tissue” is meant to refer to coronary arteries.

As used herein the terms “prior to” or “before” in the context of contacting cells (or administration of) with at least two therapeutic agents, refers to a release of a first agent at a time prior to (or overlapping with) the release of the second agent so that the release of the first agent starts before the release of the second agent. The release of the at least two agents can be achieved either in the same delivery system or in different delivery systems. For instance, in the context of a stent used as a delivery system, the stent could have multiple coatings for controlled release enabling the release of the first agent prior to the second agent.

As used herein the term “cytostatic drug” refers to, without being so limited, to paclitaxel, rapamycine, sirolimus or analogs thereof, zotarolimus, everolimus, tacrolimus, and biolimus.

One embodiment of the invention provides a method for biologically stenting a procedurally traumatized mammalian blood vessel. The method comprises administering to the blood vessel an amount of an estrogen receptor agonist in a vehicle effective to biologically stent the vessel. As used herein, “biological stenting” means the fixation of the vascular lumen in a dilated state near its maximal systolic diameter, e.g., the diameter achieved following balloon dilation and maintained by systolic pressure. The method comprises the administration of an effective amount of an estrogen receptor agonist to the blood vessel. Preferably, the estrogen receptor agonist is dispersed in a pharmaceutically acceptable liquid carrier. Preferably, a portion of the amount administered penetrates to at least about 6 to 9 cell layers of the inner tunica media of the vessel (or much deeper than that in the case of where 17 beta estradiol is used as estrogen receptor agonist) and is thus effective to biologically stent the vessel.

The present invention encompasses using in the method of the invention an estrogen receptor agonist alone or in combination with an agent able to reduce expression of an estrogen receptor beta. In specific embodiments, the agent is an antisense such as those described in U.S. Pat. No. 7,235,534 to Tanguay et al. In other embodiments, the agent is a small interference (siRNA) or a small hairpin RNA (shRNA). siRNAs and shRNAs have been successfully be used to suppress the expression of various genes in the cardiovascular field (see Dev K K. IDrugs. 2006; 9(4):279-82; Sugano M. et al, Atherosclerosis. 2007; 191(1):33-9; Takahashi et al. Biochem Biophys Res Commun. 2007; 361(4):934-40; Iantorno M. et al. Am J Physiol Endocrinol Metab. 2007; 292(3):E756-64; Platt M O et al., Am J Physiol Heart Circ Physiol. 2007; 292(3):H1479-86; Cashman S M et al., Invest Opthalmol Vis Sci. 2006; 47(8):3496-504; Hecke A. et al., Thromb Haemost. 2006; 95(5):857-64).

The present invention comprises using more than one estrogen receptor agonist. In a specific embodiment, the method uses 17 beta estradiol and an agent that blocks estrogen receptor beta.

The implantable device of the present invention possesses at least one of the following advantageous properties: accelerates reendothelialization, reduces macrophage infiltration, reduces leukocyte and/or platelet adhesion and reduction of type I collagen. This last effect allows estradiol's deep penetration and its consequent reduction of migration of smooth muscle cells from the media to the injured region exposed to blood circulation as detected in the adventitia and peri-coronary muscular and white fat tissues (FIG. 10). The adventitia includes the vasa vasorum, small vessels which provide nutriment to the vascular wall but which are also an entry door for inflammatory cells.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 presents a morphometric analysis of the stented coronary arteries 1 month post-implantation. The number of sections (n) analysed in each group is indicated between brackets;

FIG. 2 presents an immunohistology analysis of MAC-2 expression of the stented coronary arteries 1 month post-implantation. n values indicated in brackets represent number of sections analysed by group. (* p<0.05 vs Cypher™, BM and PEA group; ** p<0.05 vs 300 μg E2 group);

FIG. 3 presents the percentage of reendothelialization analysed by immunohistology of CD31 positive cells on the stented coronary artery 2 weeks post-implantation. n values indicated in brackets represent number of sections analysed by group. (* p<0.05 vs 300 ug E2);

FIG. 4 schematically shows two overlapping stents used in Examples presented herein and identifies regions of the stents: distal (Extremity 1, Ext 1), medial (Center), overlapping stent region (Overlap), and proximal (Extremity 2, Ext 2). Diagram demonstrates two overlapping stents in a coronary artery. The lengths of each stent and of the overlapping region are indicated in millimetre (mm);

FIG. 5 presents the scanning electronic microscope pictures at 30×, 100×, 500× and 2000× (in full white line box) of the different regions of an artery 1 month after the implantation of two overlapping 500 μg 17 beta-estradiol drug eluting stents. The dashed white box indicates the enlarged region;

FIG. 6 presents the scanning electronic microscope pictures at 30×, 100×, 500× and 2000× (in full white line box) of an artery 1 month after the implantation of two overlapping Taxus™stents. The dashed white box indicates the enlarged region;

FIG. 7 presents the adhesion of platelets and leukocytes on the endothelium surface of stented swine coronary segments at 1 month post-implantation. Scoring was performed as described in Example 7; excluding areas of exposed struts. For those results, three (Taxus™, Cypher™) and five (500 ug E2) stented arteries were analysed;

FIG. 8 presents scanning electronic microscope pictures at 800× of magnification of a coronary artery with exposed strut and platelets adhesion 1 month after Cypher™ stent implantation.

FIG. 9 presents 17 beta-estradiol (E2) concentration in stented coronary segments at different time points after stent implantation; three arteries were stented in two animals for each time point. RCA: right coronary artery, LAD: left anterior descending artery, LCX: circumflex. The green dashed line represents the baseline endogenous concentration of estradiol measured in coronary arteries of an untreated age-matched control animal;

FIG. 10 presents the quantification of estradiol in tissues in close proximity but not in direct contact with the 500 ug E2 releasing stents. Quantification by GC/MS of estradiol concentration reached in the adventitia of the right coronary artery, in peri-coronary muscle, in peri-coronary white fat tissues and in the apex of the heart was performed. Tissues were isolated from two animals for each time point. Values are expressed as a mean ±SEM;

FIG. 11 presents in percentage of the initial total load, the cumulative release of 17-beta-estradiol (E2) from stents at different time points after implantation in vivo. The percentage of E2 was determined using 500 μg as the reference value. Each value is the mean ±SEM of measures obtained with 3 to 6 stents; and

FIG. 12 presents the kinetic of the reduction in 17 beta estradiol (E2) concentration in stented coronary segments and stent during a two-week period.

### DESCRIPTION OF ILLUSTRATIVE EMBODIMENTS

The present invention is illustrated in further details by the following non-limiting examples.

### Example 1

### Material and Methods for Examples 2 to 4

Pre-Operative Procedures Animals were monitored and observed at least 5 days prior to experimental use. Within 24 hours prior to the procedure, the animals were started on a dosage of 650 mg aspirin, 75 mg Plavix™ and 30 mg nifedipine orally.

Stents and hormone used for the procedures The polymer poly (ester-amide) (PEA)/estradiol (E2) stents used in Examples presented herein were prepared by MicroPort (MicroPort Medical Co, Ltd. Shanghai). Stainless steel bare metal stents were of 18 mm and 23 mm length and were spray coated by MicroPort with 300 μg or 500 μg of E2 matrixed with MediVas™ poly (ester-amide) (MVPEA.I.(Ac)) tempo polymer. In addition to the polymer-steroid matrixed base coat, a 200 ug MVPEA.I.(Ac).tempo protective topcoat was applied to the stents to decrease drug elution speed. The stents were sterilized by ethylene oxide at the MicroPort Medical Co facilities under the recommendation of Medivas. The sizes of Cypher™ stents were; 3×18 mm, 3.5×18 mm, 3×23 mm, 3.5×23 mm (Johnson & Johnson). The sizes of Taxus™ stents were 2.25×12 mm, 3×16 mm, 3.5×16 mm, 3×24 mm, 3.5×24 mm (Boston Scientific). The estradiol (USP) used to load the polymer poly (ester-amide) (PEA)/E2 stents was bought from Xenex labs (Coquitlam, Canada) and send to Microport. The estradiol used for local delivery by catheter in the objective 2 was the water soluble form coupled with hydroxypropyl-heta-cyclodextrin (HPCD).

Anaesthesia At the day of procedure, animals were sedated with 1M Telazol [tiletamine and zolazepam] (6 mg/kg) mixed with Atropine (0.05 mg/kg) before transfer to the angioplasty room. The swine were intubated and ventilation started using a mixture of 70% of pure oxygen and 30% of room air. Animals were then maintained at 0.5-2% Isoflurane and supplemented with oxygen. Venous access was obtained with a 20-gauge angiocatheter in the ear vein. Maintenance intravenous hydration was provided with 0.9% sodium chloride solution at a rate of 10 ml/kg/hr, including estimated blood loss.

Catheterization Following induction of anaesthesia, an 8 French arterial sheath was introduced in the right or left femoral artery. A 7 French or 8 French large lumen guiding-catheter was placed into the sheath and advanced via a 0.035″ guide wire under fluoroscopic guidance into the appropriate coronary arteries. After placement of the guiding catheter into the coronary artery, angiographic images of the vessel were obtained to identify the proper location and size for the deployment site using Quantitative Coronary Angiography (QCA) method.

Post-operative Procedures Immediately following the procedure, the femoral sheath was removed and compression was done to obtain complete haemostasis. The swine were allowed to recover in the transport pen under observation before be returned to their usual pens. Plavix™ 75 mg SID (once a day) was administered during follow-up.

Follow-up Procedures and Termination For animals used in Examples presented herein, 24 h, 14 days, 1 month and 3 months post-procedure, animals were re-anaesthetized and an arterial stick was inserted in the right or left femoral artery. A 7 or 8 French guiding catheter was placed into the sheath and advanced via a 0.035″ guide wire under fluoroscopic guidance into the treated vessels. After placement of the guiding catheter into the artery, angiographic images of the vessels were taken to evaluate the treatment site by QCA measurements. Animals used in Examples 1 and 2 were euthanized and stented segments were perfusion-fixed ex vivo with 10% buffered formalin at 100 mmHg. The stented vessels were harvested and transferred to the Histology Laboratory for complete histological analysis.

Statistical Analysis Statistical analysis was performed using commercial software (Primer of Biostatistics™, version 3.0). Differences between groups were determined by ANOVA™ followed by a Bonferroni's test correction for multiple comparisons. A p-value<0.05 was considered as statistically significant. Values were expressed as mean ±standard error of the mean (SEM).

Compiled results and analysis All the swine had comparable weight (Wt) at baseline and have shown a normal growth and weight gain in time. The procedural peak Activated Clotting Time (ACT) was greater than 300 seconds in order to successfully perform stent implantation. Blood pressure and heart rate were within physiological limits. Blood analysis at baseline and follow up were normal for the levels of white blood cells (WBC), red blood cells (RBC), hemoglobin (Hb), hematocrit (Hct) and platelet (Plt).

Quantitative coronary angiography (QCA) was performed pre and post stent deployment, and before sacrifice at each target vessel including proximal and distal reference segments. Each angiography was performed after intra-coronary administration of 100-200 μg nitroglycerin. Angiographic patency and the physiological responses of the vessels to all implanted stents were evaluated at 24 hours, 14 days, 1 and 3 months by QCA analysis. A balloon to artery ratio (B/A ratio) of 1.1 to 1.2:1 was achieved in all groups. No significant difference was observed in vessel diameter of the proximal (Prox) and distal (Dist) reference segments, and of the treated (Tx) coronary segment at baseline, after balloon dilation or immediately post stent implantation.

### Example 2

### Effect of 16.7 μg/mm (300 μg on 18 mm stent) and 27.8 μg/mm (500 μg on 18 mm of Stent) 17 Beta-Estradiol on Neointima Formation

Animal groups Vascular healing (efficacy study) was evaluated in six animals for each type of stent (bare metal stent (BM), polymer poly (ester-amide) (PEA) only, PEA with 300 μg E2, PEA with 500 μg E2, at 24 hours, 14 days, 1 and 3 months after the stent implantation and 2 animals for Cypher™ and Taxus™ at 1 month post-implantation.

Stents Three stents were implanted in each animal. All animal groups were successfully completed and survived until the pre-determined time point.

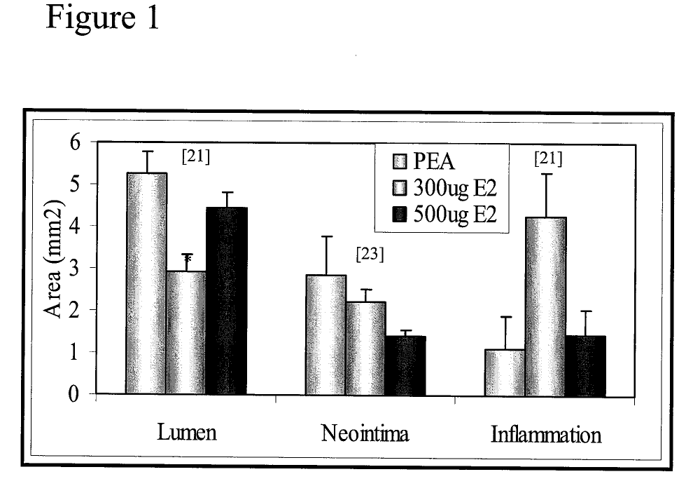

Morphometric analysis was performed for each stented artery. Measurements of the neointima area (Neointima), the lumen area (Lumen), media area (Media) (i.e. thickness of intima and adventitia together), and the percentage of stenosis are summarized for control bare metal stent (BM), PEA polymer only (PEA), coated with PEA+300 μg of E2 (300 μg E2) or PEA+500 μg of E2 (500 μg E2) treatments) in 1 month groups in Table 1. Histogram compilation of the neointima, inflammation and lumen area for stent coated with PEA, PEA+300 μg of E2 (300 μg E2) or PEA+500 μg of E2 (500 μg E2) of 1 month group is presented in FIG. 1. At the follow-up, in the four groups, all the stents were patent (i.e. not occluded).

At 1 month post-procedure, the 300 μg E2 and 500 μg E2 groups presented no significant difference in the degree of Ni formation with the BM, PEA, Taxus™ and Cypher™ groups (FIG. 1 and Table 1 below). However, the 300 μg E2 group had a more pronounced reduction in lumen area compared to the other groups and an increased percentage of stenosis. These two effects could largely be due to an ongoing inflammation process that is unexplained. Note that, without being bound by this particular theory, this type of phenomenon was reported previously and could be caused by the presence of biological (bacteria) or chemical contaminants in the poly(ester-amide) polymer. Possible alterations in the degradation process of the PEA due to the sterilization process (ethylene oxide) cannot be excluded. This inflammation process was distinct of the Ni. At this time point, the high dose 500 μg E2 group has the same percentage of stenosis as the Taxus™ and Cypher™ groups and less expansion in total vessel area than the PEA group. The unusual inflammation was not detected in the BM, Taxus™ and Cypher™ groups (Table 1 below).

TABLE 1
Morphometric measuments of 1-month groups.

| | | | | | | | | | |
|---|---|---|---|---|---|---|---|---|---|
| | T area | INJ | Ni thickness | L area | Ni area | M area | Inf | Inf | Stenosis |
| Tx (n) | mm² | score | mm | mm² | mm² | mm² | mm² | % | % |

A.

| | | | | | | | | | |
|---|---|---|---|---|---|---|---|---|---|
| BM (24) | 8.28 ± 0.39 | 2.37 ± 0.09 | 0.25 ± 0.02 | 5.83 ± 0.36 | 1.28 ± 0.14 | 1.17 ± 0.08 | 0 | 0 | 18.84 ± 2.27 |
| PEA (21) | 9.97 ± 0.71 | 2.68 ± 0.12 | 0.31 ± 0.03 | 5.26 ± 0.50 | 2.86 ± 0.93 | 0.93 ± 0.11 | 1.11 ± 0.77 | 6.83 ± 4.70 | 22.74 ± 4.01 |
| 300 μg E2 (23) | 11.03 ± 0.73 | 2.36 ± 0.13 | 0.40 ± 0.04 | 2.93 ± 0.40† | 2.22 ± 0.30 | 0.61 ± 0.16 | 4.25 ± 1.05* | 33.64 ± 7.77* | 43.96 ± 5.07 |
| 500 μg E2 (21) | 8.01 ± 0.56 | 2.74 ± 0.10 | 0.28 ± 0.02 | 4.46 ± 0.37 | 1.39 ± 0.18 | 0.76 ± 0.12 | 1.43 ± 0.60 | 12.81 ± 5.29 | 24.87 ± 3.25 |

B.

| | | | | | | | | | |
|---|---|---|---|---|---|---|---|---|---|
| Taxus (8) | 7.83 ± 0.38 | 2.64 ± 0.13 | 0.29 ± 0.03 | 5.38 ± 0.37 | 1.57 ± 0.13 | 0.88 ± 0.05 | 0 | 0 | 23.66 ± 2.55 |
| Cypher (14) | 7.43 ± 0.27 | 2.77 ± 0.10 | 0.3 ± 0.02 | 5.01 ± 0.27 | 1.52 ± 0.13 | 0.89 ± 0.06 | 0 | 0 | 23.56 ± 2.02 |

Tx: treatment,
T: total artery,
INJ: injury,
Ni: neointima,
L: lumen,
M: media,
Inf: inflammation,
mm: millimeter.
*p < 0.05 vs PEA group;
†p < 0.05 vs BM and PEA groups.

### Example 3

### Effect of 16.7 μg/mm (300 μg on 18 mm stent) and 27.8 μg/mm (500 μg on 18 mm of Stent) 17 Beta-Estradiol on Macrophage Infiltration

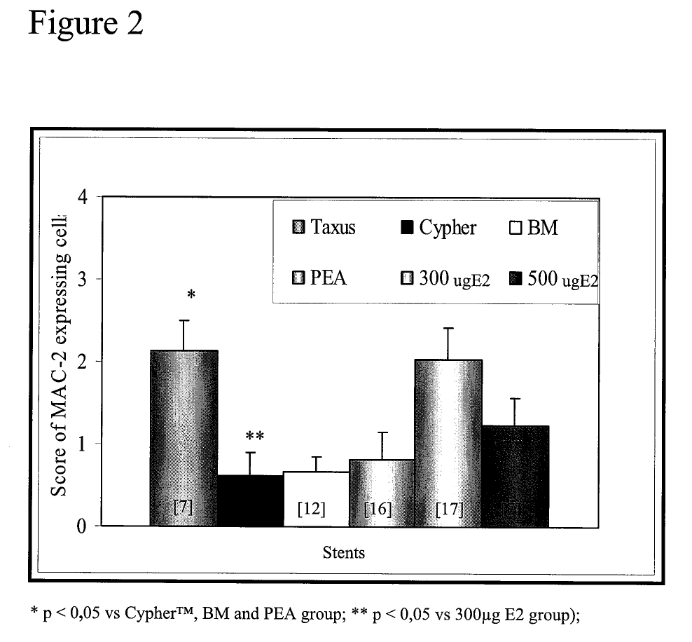

Immunohistology analysis was performed to determine the degree of macrophage infiltration in the wall of the stented arteries. Macrophage infiltration is used as an inflammation marker and as an indication of risk of thrombosis. Macrophage infiltration was evaluated using an anti-MAC-2 specific antibody (MAC-2), a cell surface marker for macrophages. Macrophage infiltration was scored for each strut as follow: 0: absence of macrophages, 1: very rare number of macrophage, 2: limited number macrophages, 3: high number of MAC-2 positive cells, 4: very high number of macrophages around the strut and between the struts. The mean of scores of the struts corresponds to the score for the artery. As may be seen in FIG. 2, after 1 month, the 300 μg E2 group had more macrophages (mean grade of 2) than three other groups but it is comparable to infiltration levels observed with the Taxus™ stents. The 500 ug E2 group had an infiltration level not statistically different to the Cypher™ stents (see FIG. 2).

### Example 4

### Effect of 16.7 μg/mm (300 μg on 18 mm stent) and 27.8 μg/mm (500 μg on 18 mm of Stent) 17 Beta-Estradiol on Reendothelialization

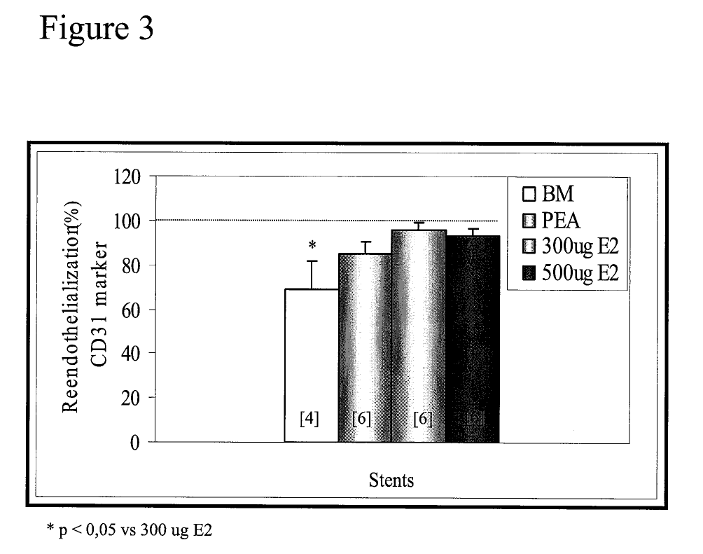

Evaluation of the reendothelialization process was also performed by immunohistology at the different time points. Arterial histology slides were stained with an anti-CD31 antibody, a cell surface marker for endothelial cells. The percentage of the intima positive for CD31 was determined for each artery and compiled results are presented in FIG. 3. After two weeks, the reendothelialization process was completed by >90% in the 300 μg and 500 μg E2 groups while it was of 70% to 85% in the BM and PEA groups, respectively. This result suggests a faster reendothelialization process in the presence of estradiol.

### Example 5

### Material and methods for Examples 6 to 7

Animal groups and samples analyzed A total of 7 swine were distributed into 3 groups defined on the type of stents/treatments received; 1) PEA/500 μg E2 stents (N=3), 2) Cypher™ (N=2) or 3) Taxus™ (N=2). All animal groups were successfully completed and survived until the pre-determined time point.

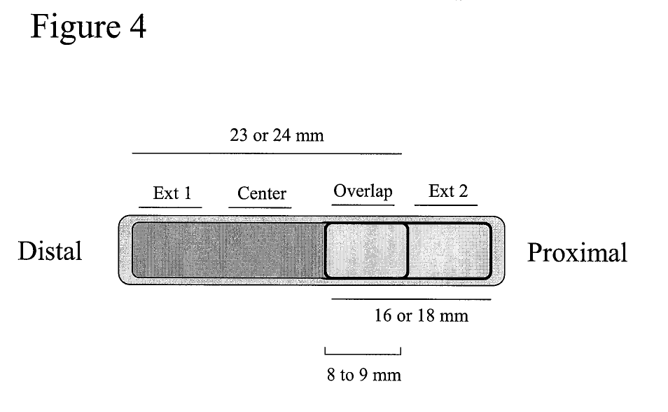

Stents Swine were implanted with 4 stents (2 single stents and 2 overlapping stents). Overlaps were performed with 23 mm long stents (for a dosage of 17 beta-estradiol on the single portion of about 21.7 μg/mm) and 18 mm long stents (for a dosage of 17 beta-estradiol on the overlap of about 27.8 μg/mm) overlapped over a 9 mm segment (for a dosage of estradiol on the overlap of about 49.5 μg/mm) (FIG. 4). Single stents were 18 mm long (for a dosage of estradiol on the overlap of about 27.8 μg/mm). One Cypher™ stent did not deploy properly and was therefore removed from one animal. In this animal, two single non-overlapped stents were implanted in the RCA.

The stented segments and the proximal and distal regions of the arteries were dissected from the formalin-fixed hearts and open longitudinally. A total of 20 stents were recovered. For the two groups with PEA/E2 stents, 2 singles stented arteries and 1 arteries stented with overlapped stents were processed for SEM analysis. In the Cypher™ and the Taxus™ groups, 2 single stented arteries and 1 overlap were analyzed in SEM. The other stented segments were processed for morphometry analysis.

Compiled results and analysis For each stented artery, a picture was taken in the distal (Extremity 1, Ext 1), medial (Center) and the proximal (Extremity 2, Ext 2) region at 30×, 100×, 500× and 2000× using a scanning electronic microscope (FEGSEM, model S-4700 from Hitachi). In the case of the overlapped stents, an additional picture was taken in the middle of the region of the overlap (named Overlap). The Center picture corresponds to the non-overlapped region of the 23 mm or 24 mm stent (See FIG. 4).

### Example 6

### Effect of 21.7 μg/mm of Stent Length (500 μg on 23 mm Stent), 27.8 μg/mm of Stent Length (500 μg on 18 mm of Stent) and 49.5 μg/mm of Stent Length (Overlap of 500 μg on 23 mm of Stent Length and 500 μg on 18 mm of Stent Length) 17 Beta-Estradiol on Reendothelialization and Neointima Formation

Qualitative evaluation of arterial wall thickness and state of reendothelialization was performed and the results after one month are presented in Table 2 below. This table includes the mean value of arterial thickness for the three or four segments (Ext1, Center, Overlap, Ext 2) of each artery. Wall thickness classification used was; −: no Ni visible, +: small Ni or Ni limited to only one region ++; important Ni but the lumen area is larger than the lumen area outside the stented segment, +++: large Ni all along the segment with important reduction of the lumen area.

TABLE 2
Reendothelialization process in arteries of 1-month groups

| | | | | |
|---|---|---|---|---|
| | | Wall | | |
| Swine | Artery | Thickness | Reendothelialization | Comments |

PEA/E2 500 μg E2

| | | | | |
|---|---|---|---|---|
| Est-49 | LCX | ++ | Complete | |
| Est-49 | RCA | +++ | Complete | |
| Est-50 | LAD | + | Complete | large plaque |
| Est-50 | RCA | + | Complete | |
| Est-51 | LCX | +++ | Complete | denuded region due to the |
| | | | | procedure |

Taxus ™

| | | | | |
|---|---|---|---|---|
| Est-55 | LAD | − | (Complete), Thin, (cracks) | Majority of cracks due to the |
| | | | | procedure |
| Est-55 | RCA | − | Complete, Thin | Cracks due to the procedure |
| Est-57 | RCAprox | + | Near Complete, Thin, Cracks | No tight junction between EC |

Cypher ™

| | | | | |
|---|---|---|---|---|
| Est-56 | LCX | + | Complete, Thin | |
| Est-56 | RCA | − | Incomplete, Thin, Expose strut | |
| Est-58 | LAD | − | (Complete), Thin | Small holes in all the stented |
| | | | | segment |

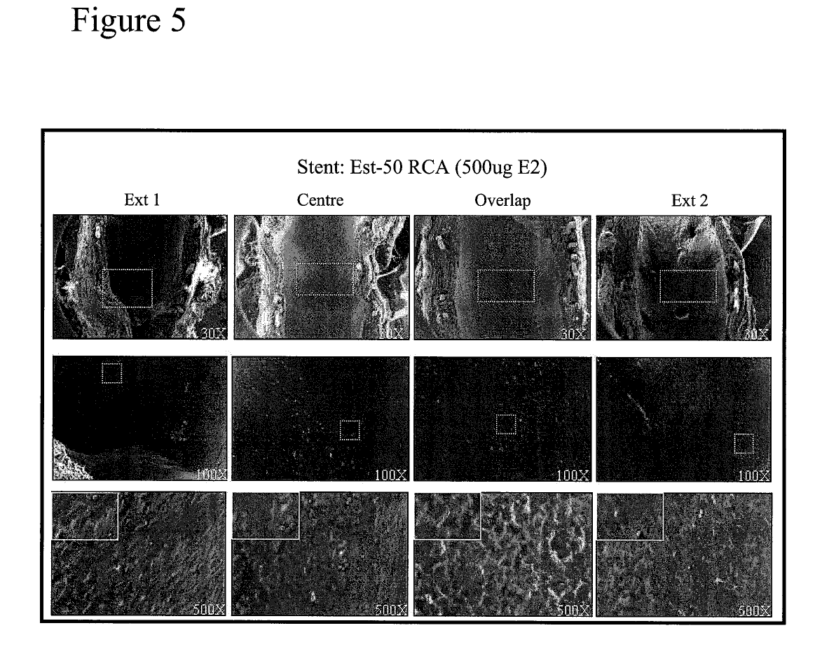

One month after implantation of PEA 500 μg E2 stents, a complete reendothelialization of the stented region is observed over the Ni (see FIG. 5 for a representative SEM picture for PEA 500 μg E2 stents).

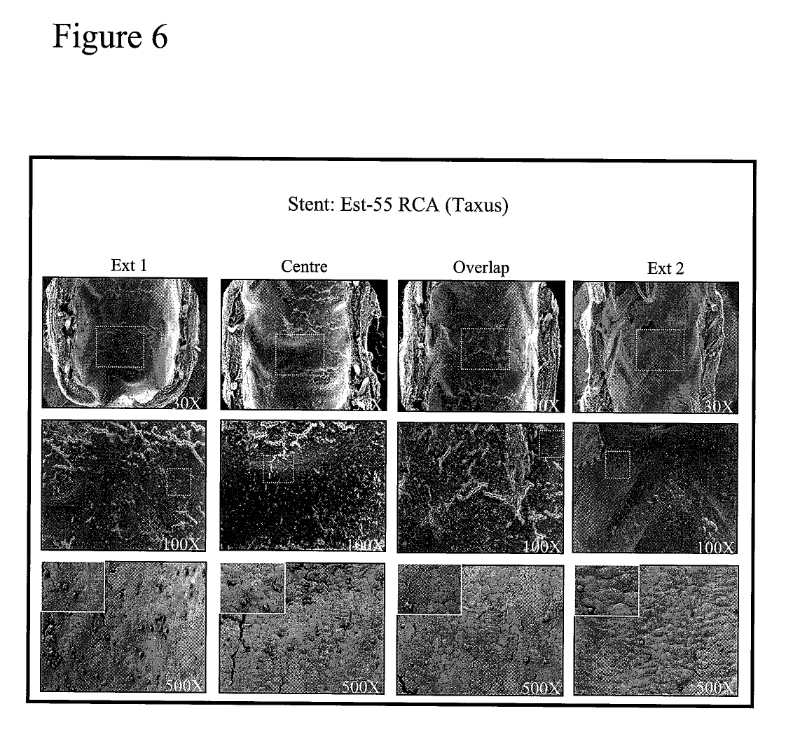

In arteries stented with Taxus™ or Cypher™, no or minor Ni developed (see FIG. 6 for a representative SEM picture for Taxus™ stent). The newly formed endothelium is thin however with strut design easily visible. One of the three Cypher™ stents had multiple metal struts exposed to blood flow.

### Example 7

### Effect of 21.7 μg/mm (500 μg on 23 mm Stent), 27.8 μg/mm (500 μg on 18 mm of Stent) and 49.5 μg/mm (Overlap of 500 μg on 23 mm Stent and 500 μg on 18 mm of Stent) 17 Beta-Estradiol on Number of Platelets/Leukocytes on Stented Coronary Segments

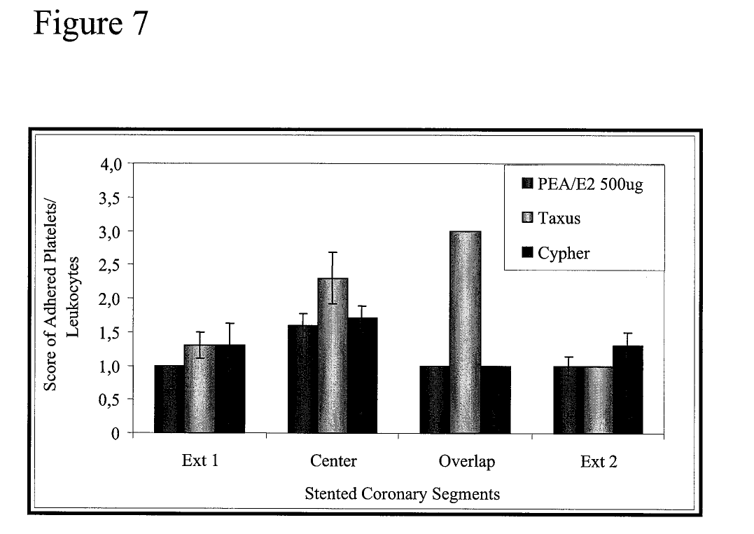

The presence of platelets is an indication of activated endothelium (i.e. an endothelium that expresses certain markers following an injury) while the presence of leucocytes is an inflammation marker. A qualitative evaluation of the number of platelets/leukocytes adhered to the lumen side of the artery was graded as follow; 0: absence of adhered cells, 1: cells are usually dispersed and rare, 2: significant amount of cells (more than 40 cells in the 500× magnified region), 3: high number of cells on most of the surface (evaluated with the 100× magnified field) with presence of multiple grouped platelets, 4: very high number of cells with large aggregates (thrombus). Results are summarized in FIG. 7. PEA 500 ug E2 and the Cypher™ stents show very comparable number of adhered platelets/leukocytes with maximum number located in the center region. Also of interest is the fact that the overlap region of PEA 500 ug E2 in FIG. 7 is as good or better than the single stent regions. Indeed the double layer of metal usually presents an increased risk for Ni and thrombosis and this Figure shows that the PEA 500 ug E2 was able to prevent this risk.

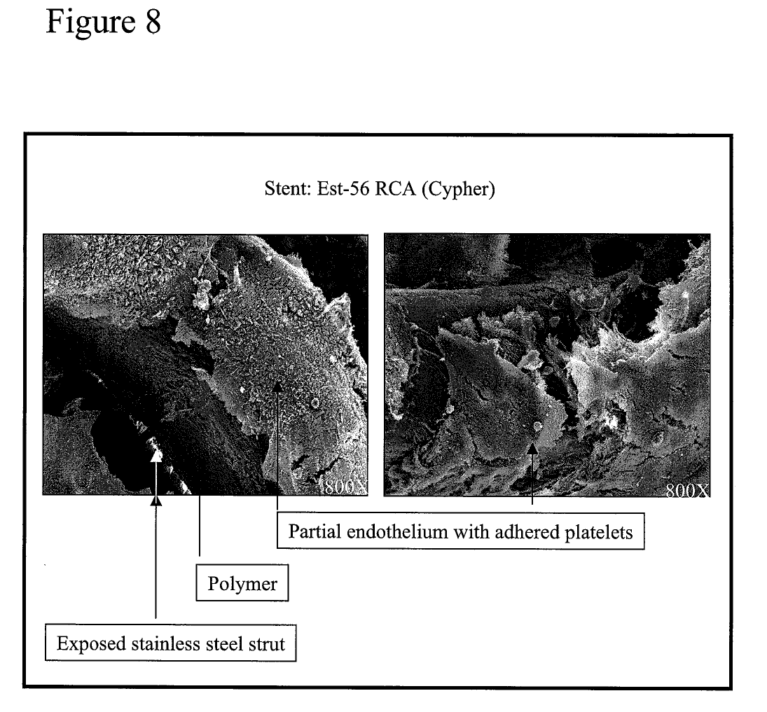

Overall, in the three groups, the number of leukocyte and platelets was very low when compared with pro-thrombotic regions such as exposed strut of the Cypher™ (See FIG. 8, Est-56 RCA, magnification 800×). Exposed strut regions are known to be pro-thrombotic. The PEA+500 ug stents did not present any exposed struts.

### Example 8

### In Vivo Release Study

Animal groups The protocol was successfully completed in all animals for each group. Animals were euthanized at the predetermined time-points (1, 3, 6, 24 and 48-hour, 1 and 2-week). A total of 42 stents were recovered from 14 swine (3 stents/animal) excluding 1 swine which died prematurely at day 5 post-implantation.

Stents The stents used for this study were MicroPort™ stents (MicroPort Medical Co, Ltd. Shanghai) of 18 mm length spray coated by Microport with 500 ug of E2 matrixed with MediVas poly(esteramide) (MVPEA.I.(Ac)) tempo polymer. In addition to the polymer-steroid matrixed base coat, a 200 μg MVPEA.I.(Ac).tempo protective topcoat was applied to the stent to prevent rapid drug elution. The stents were sterilized by ethylene oxide at the Microport Medical Co facilities under the recommendation of Medivas.

Tissue samples and stents were analyzed for each animal for estrogen content. The right coronary artery (RCA) and the left anterior descending artery (LAD) were opened longitudinally and cut into 6 pieces; 2 in the proximal region, 2 in the medial region and 2 in the distal region of the stented segment. One piece in each region was analyzed and the other one was kept as a backup. To evaluate the migration of E2 through the arterial wall, the left circumflex artery (LCX) was dissected to separate the adventitia from the rest of the artery and the two fractions were analyzed individually. A total of 42 stents were recovered from 14 swine (3 stents/animal). After centrifugation, sera were collected for extraction and analysis by GC/MS.

Hormones Deuterium-labeled estradiol 2,4,16,16,17-D5 (17β-D5) (CDN Isotopes, Pointe Claire, Canada) and micronized estradiol (USP, Xenex labs, Coquitlam, Canada) were used respectively as internal and external controls.

Extraction of steroids—17β-estradiol extraction in pig tissues and in coated stents. Stents were dissected from the coronaries before the extraction procedure. For the extraction of estradiol in pig tissues, up to 100 mg of frozen tissues were crushed before addition of 3 mL of a solution of MeOH-chloroform (2:1). For the extraction in the coated stents, the coating was dissolved in 3 mL of dichloromethane. A solution of 17β-D5 was added to all samples as an internal standard. After centrifugation, the supernatant was dried over Na₂SO₄ before evaporation under a stream of N₂.

Extraction of steroids—17β-estradiol extraction in pig serum. Blood samples of 8.5 mL were collected at specific time from each animal in the coronary sinus using an 8 French guiding catheter at baseline, 10 min and 1 hr after stent implantation and at sacrifice. Samples were centrifuged at 1800 rpm at 4° C. for 10 minutes. Serum was collected and 500 μL were mixed with 17β-estradiol-d₅ as an internal standard as well as a solution of MeOH/H₂O (80:20). As was also done with the tissue samples, the serum samples were sonicated then centrifuged. The supernatant was transferred into a new tube. Acetate buffer 0.2M was added to the tubes and a double liquid-liquid extraction with ethyl ether was performed. Samples were dried over Na₂SO₄ before evaporation under nitrogen (N₂).

Derivatization and GC-MS assays. Evaporated samples were reconstituted in a pyridine solution before addition of a pentafluorobenzoyl chloride derivative (PFBCI) solution. Derivatization was performed at 60° C. Once the reaction was completed, a back-extraction was performed with a solution of NaHCO₃ to stop the reaction before a liquid-liquid extraction. Samples were dried over Na₂SO₄ before complete evaporation under a stream of N₂. Samples were reconstituted in iso-octane, final volume was adjusted to 50 μL and samples were injected in bench-top standard GC-MS equipment (Agilent Technologies) gas chromatograph coupled to a 5973 Mass Selective Detector™. The injections, performed by a Model 7683 Series Injector, were of 2 μL in the pulsed-splitless mode. The carrier gas was high-purity helium. An Agilent Technologies™ type DB-17ht capillary column was used. Ions 460 and 465, which corresponds to the [M-H]⁺ ions of 17β-estradiol and of internal standard [²H₅]17β-estradiol respectively, were monitored with a dwell time of 50 ms per ion.

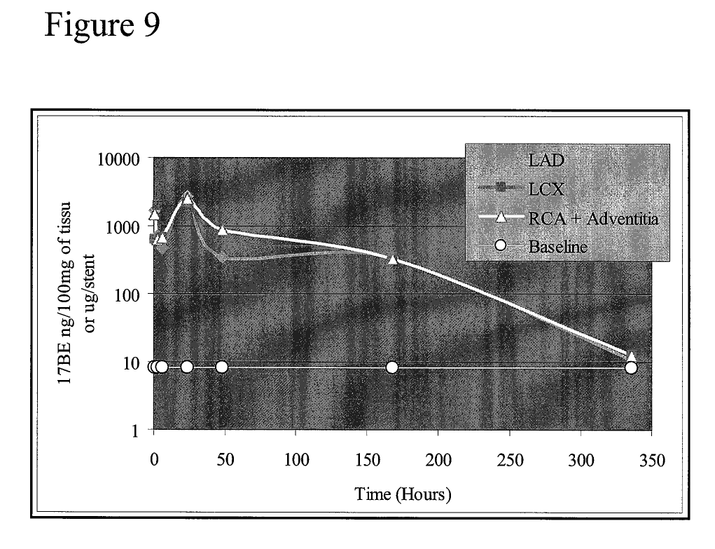

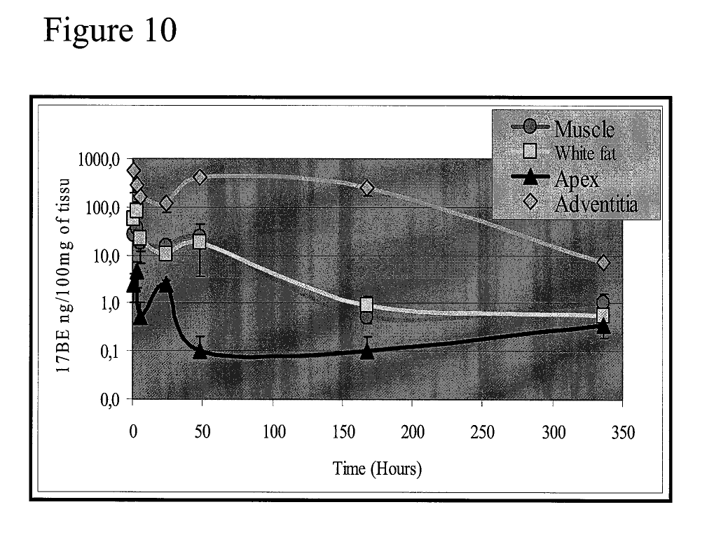

Estrogen quantification data Estrogen quantification was performed by Gas Chromatography/Mass Spectroscopy (GC/MS) as described above. Diffusion of E2 from the stent was determined in tissue in direct contact with the stent (coronary tissues proximal, medial, distal (see FIG. 9)), in close proximity (reference proximal, reference distal, adventitia, peri-coronary muscle, peri-coronary white fat (see FIG. 10)) or at distal sites potentially exposed to E2 released from the stent into the blood (carotid artery, femoral artery, apex, auricle, lung, kidney and liver). To obtain the total amount of E2 in an artery, E2 content measured by GC/MS in the proximal, medial and distal portions of each stented coronary segment were added together. Because the arteries were subdivided in two longitudinal cuts, the value obtained was multiplied by two to extrapolate the concentration in the complete artery segment. The E2 content of the total adventitia dissected from each RCA was also determined.

FIG. 9 shows that the mean concentration of E2 maintained in the coronary tissues was of about 10.6 μg/g of tissue during the first week after administration, and of 12.0 μg/g of tissue after 48 hours of administration.

It also is interesting to note that estradiol could achieve deep penetration. This is advantageous because some inflammatory cells penetrate the arterial wall via the vasa vasorum of the adventitia, namely in the periphery of the vessel. This resulted in a reduction of migration of smooth muscle cells from the media to the injured region exposed to blood circulation as detected in the adventitia and peri-coronary muscular and white fat tissues (FIG. 10).

E2 concentration in tissues at distal location from stent are in all cases, at baseline level (endogenous E2), a value determined from the same tissues isolated from swine not treated with E2. At all studied times, E2 released from the stent in the blood stream was not sufficient to affect E2 concentration at distal sites. Enhancement of E2 concentration in sera over the baseline level was detected in 5 of the 14 animals. Elevated E2 levels in the blood were still detected 24 h and even 1 week after stent implantation. This could be an indication of variations of E2 content among stents or of the kinetic in E2 release or a combination of both.

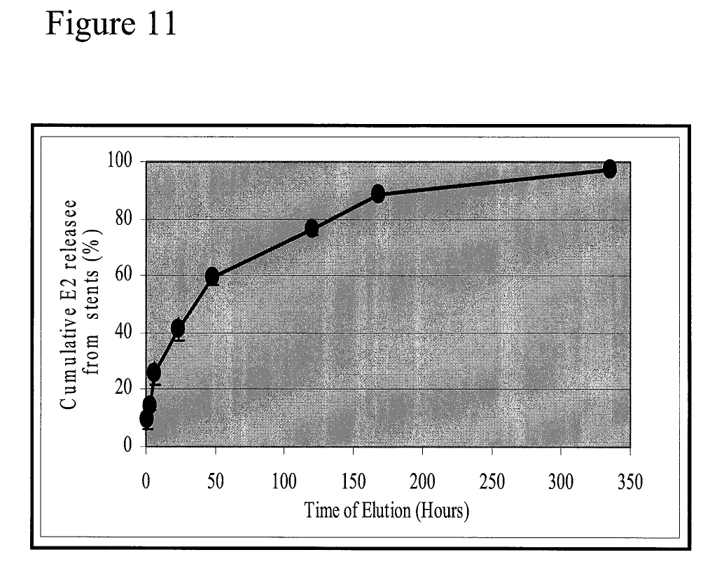

In parallel to the quantification of E2 in tissues and blood, E2 concentration remaining on the stent at different time points after implantation was also determined. Stents were dissected from LAD, LCX and RCA arteries and E2 extracted for quantification by GC/MS. Results are presented in cumulative percentage of E2 released from stents at the different time points (FIG. 11).

The percentage of E2 was determined using 500 μg as the reference value. However, the real amount of E2 on each stent was known to be variable as measured in 3 intact un-deployed E2 500 μg-stents. The mean ±SEM E2 concentration/stent measured was 554±28 μg (namely about 30.8 μg/mm of length of stent). In conclusion, 90% of E2 was eluted from the stent after 1 week, with 50% released in the first 48 hours (FIG. 11). It is expected that increasing the top coat thickness will alleviate this problem. It is also important to note that the healing process is quicker in pigs than it is in humans (a few months in humans compared to 1 month in pigs) so that the release of the estradiol dose according to the present invention is desirably extended over a few weeks.

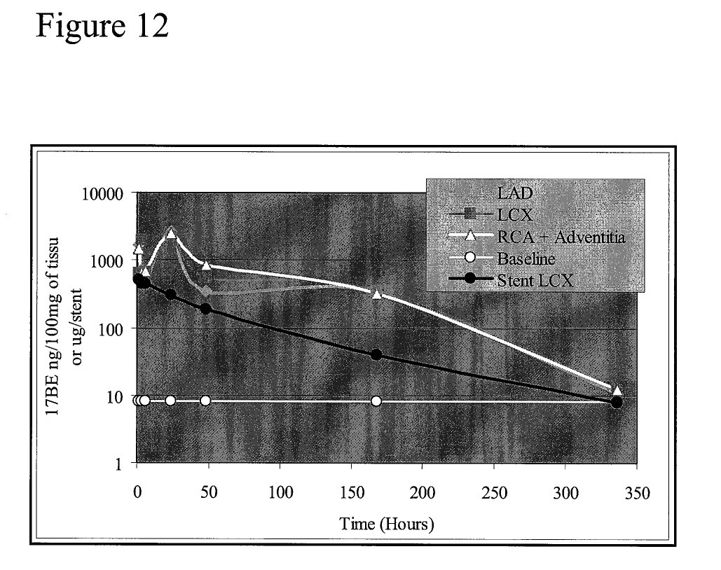

Kinetics of the reduction in E2 concentration in stented coronary segments and stents during a two-week period was compiled on the same graph to facilitate the comparison between their respective curves (FIG. 12). It is important to notice that measure units for E2 in tissues and in stents are different.

### Example 9

### Implantation of Stent in Patient

Before stent implantation, the obstructed artery is usually dilated using an angioplasty balloon. Then a stent of the appropriate size is guided to the targeted site. The balloon is inflated to deploy the stent with complete apposition to the arterial wall. The angioplasty balloon is deflated and removed.

Although the present invention has been described hereinabove by way of specific embodiments thereof, it can be modified, without departing from the spirit and nature of the subject invention as defined in the appended claims.

### REFERENCES

1. Chandrasekar B. et al. Local delivery of 17β-estradiol improves reendothelialization and decreases inflammation after coronary stenting in a porcine model. Thromb Haemost 2005; 94:1042-7.
2. Chandrasekar B et al. Coronary artery endothelial protection after local delivery of 17β-estradiol during balloon angioplasty in a porcine model: A potential new pharmacologic approach to improve endothelial function. J Am Coll Cardio 2001; 38(5):1570-6.
3. Abizaid A et al. First human experience with the 17-beta-estradiol-eluting stent: the Estrogen And Stents To Eliminate Restenosis (EASTER) trial. J Am Coll Cardiol. 2004 Mar. 17; 43(6):1118-21.
4. Hodgson et al. Late Stent thrombosis: Considerations and practical advice for the use of drug-eluting stents: a report from the Society for Cardiovascular Angiography and Interventions Drug-eluting Stent Task Force. Catherization and Cardiovascular Interventions 69: 000-000 (2007).
5. Finn A V et al. Pathological correlates of late drug-eluting stent thrombosis: strut coverage as a marker of endothelialization. Circulation. 2007 May 8; 115(18):2435-41.
6. Kotani K et al. Incomplete neointimal coverage of sirolimus-eluting stents: angioscopic findings. J Am Coll Cardiol. 2006 May 16; 47(10):2108-11.
7. Dev K K. Using RNAi in the clinic. IDrugs. 2006 April; 9(4):279-82.
8. Sugano M. et al, SiRNA targeting SHP-1 accelerates angiogenesis in a rat model of hindlimb ischemia Atherosclerosis. 2007 March; 191(1):33-9.
9. Takahashi et al. Functional role of stromal interaction molecule 1 (STIM1) in vascular smooth muscle cells. Biochem Biophys Res Commun. 2007 Oct. 5; 361 (4):934-40.
10. Iantorno M. et al., Ghrelin has novel vascular actions that mimic P13-kinase-dependent actions of insulin to stimulate production of NO from endothelial cells Am J Physiol Endocrinol Metab. 2007 March; 292(3):E756-64.
11. Platt M O et al., Expression of cathepsin K is regulated by shear stress in cultured endothelial cells and is increased in endothelium in human atherosclerosis Am J Physiol Heart Circ Physiol. 2007 March; 292(3):H1479-86.
12. Cashman S M et al., Inhibition of choroidal neovascularization by adenovirus-mediated delivery of short hairpin RNAs targeting VEGF as a potential therapy for AMD. Invest Opthalmol Vis Sci. 2006 August; 47(8):3496-504.
13. Hecke A. et al., Successful silencing of plasminogen activator inhibitor-1 in human vascular endothelial cells using small interfering RNA. Thromb Haemost. 2006 May; 95(5):857-64.

1. An implantable device for the controlled delivery of an estrogen receptor agonist to an injured site in the lumen of a mammalian blood vessel, wherein the estrogen receptor agonist is present in an amount of at least about 16.7 μg/mm implantable device length.

2. The implantable device of claim 1, wherein the blood vessel is procedurally traumatized.

3. The implantable device of claim 1 wherein the estrogen receptor agonist is present in an amount of at least about 21.7 μg/mm of device length.

4. The implantable device of claim 1 wherein the estrogen receptor agonist is present in an amount of at least about 27.8 μg/mm of device length.

5. The implantable device of claim 1 wherein the estrogen receptor agonist is present in an amount of at least about 30.8 μg/mm of device length.

6. The implantable device of claim 1 wherein the estrogen receptor agonist is present in an amount of at least about 49.5 μg/mm of device length.

7. The implantable device of claim 4, wherein the controlled delivery enables a mean concentration of estrogen receptor agonist in coronary tissue of at least about 9.1 μg/g of coronary tissue over at least about 2 weeks.

8. The implantable device of claim 4, wherein the controlled delivery enables a mean concentration of estrogen receptor agonist in coronary tissue of at least about 10.6 μg/g of coronary tissue over at least about 1 week.

9. The implantable device of claim 4, wherein the controlled delivery enables a mean concentration of estrogen receptor agonist in coronary tissue of at least about 12 μg/g of coronary tissue over at least about 48 hours.

10. The implantable device of claim 4, wherein the controlled delivery enables a mean concentration of estrogen receptor agonist in coronary tissue of at least about 13.6 μg/g of coronary tissue over at least about 24 hours.

11. The implantable device of claim 4, wherein the controlled delivery enables a mean concentration of estrogen receptor agonist in coronary tissue of at least about 50 μg/g of coronary tissue over at least about 24 hours.

12. The implantable device of claim 1, which is a stent.

13. The implantable device of claim 1, which is a shunt.

14. The implantable device of claim 1, which is a mesh.

15. The implantable device of claim 1, which is an artificial graft.

16. The implantable device of claim 1, wherein the estrogen receptor agonist is 17-β estradiol.

17. The implantable device of claim 16, wherein the 17-β estradiol amount is sufficient to allow complete reendothelialization of the injured site.

18. The implantable device of claim 1, wherein the estrogen receptor agonist is releasably embedded in, coated on, or embedded in and coated on, the device.

19. The implantable device of claim 1, wherein the device further comprises a controlled release polymer coating.

20. The implantable device of claim 19, wherein the polymer coating is biodegradable.

21. The implantable device of claim 1, further comprising an agent selected from the group consisting an antioxidant, an anti-proliferative/cytostatic agent, an anti-inflammatory/immunomodulator agent, an anti-migration agent and a pro-healing agent, and combinations thereof.

22. The implantable device of claim 21, wherein the cytostatic agent is paclitaxel or an analog thereof.

23. The implantable device of claim 21, wherein the cytostatic agent is rapamycin or an analog thereof.

24. The implantable device of claim 21, wherein the cytostatic agent is sirolimus or an analog thereof.

25. The implantable device of claim 21, wherein the anti-migration agent inhibits migration of vascular smooth muscle cells.

26. The implantable device of claim 25, wherein the anti-migration agent is cytochalasin.

27. The implantable device of claim 1, wherein the mammalian blood vessel is a human blood vessel.

28. A method for promoting reendothelialization of an injured blood vessel of a subject, comprising implanting the implantable device of claim 1 at an injured site of the blood vessel of the subject, whereby reendothelialization is promoted.

29. The method of claim 28, wherein the device is implanted before, during or after vascular injury, or any combination thereof.

30. The method of claim 28, wherein the injured blood vessel is a procedurally traumatized blood vessel.

31. The method of claim 28, wherein the subject is human.
